(19) United States
(12) Patent Application Publication
(10) Pub. No.: US 2026/0269060 A1
(43) Pub. Date: Sep. 10, 2026

(54) SYSTEMS AND METHODS FOR AI-DRIVEN TELEMEDICINE CONSULTATION ASSISTANT WITH AUTOMATED PATIENT TRIAGE

(71) Applicant: Dana Bradshaw, Cypress, TX (US)

(72) Inventor: Dana Bradshaw, Cypress, TX (US)

(21) Appl. No.: 19/072,534

(22) Filed: Mar. 6, 2025

(51) Int. Cl. G16H 50/20; G06F 40/35; G16H 10/60; G16H 70/20; G16H 80/00

(52) U.S. Cl. CPC G16H 50/20; G16H 10/60; G16H 70/20; G16H 80/00; G06F 40/35

(57) ABSTRACT

An AI-driven Telemedicine Consultation Assistant (AI-TCA) is disclosed, providing automated preliminary consultations using NLP, decision trees, machine learning, and EHR integration. The system analyzes patient symptoms, medical history, and lab results to offer diagnostic recommendations, triage advice, and health insights, enhancing telemedicine efficiency and patient engagement.

### TECHNICAL FIELD

The embodiments provided herein relate to systems and methods for providing an AI telemedicine consultation assistant.

### BACKGROUND

Telemedicine is revolutionizing healthcare by facilitating remote consultations, but existing systems largely rely on human physicians to triage, diagnose, and offer healthcare recommendations. This process can lead to inefficiencies, such as long wait times and underutilization of healthcare professionals. Furthermore, telemedicine systems often lack automated, intelligent consultation tools that can offer personalized care prior to direct interaction with a healthcare provider.

Current telemedicine solutions primarily rely on scheduled video calls between patients and healthcare providers. While these platforms offer convenience, they do not fully address the challenges of healthcare accessibility, especially in areas with provider shortages. Additionally, many existing platforms employ basic symptom checkers that follow rule-based algorithms rather than advanced AI-driven decision-making, limiting their ability to provide accurate and context-aware recommendations.

Another major shortcoming in the industry is the lack of effective integration between telemedicine platforms and Electronic Health Records (EHR). Many systems fail to retrieve and utilize patient history, lab results, and past diagnoses efficiently. As a result, patients often need to manually provide their medical information, leading to incomplete assessments and potential misdiagnoses. This disconnect can delay treatment, reduce the accuracy of triage, and create inefficiencies in patient care management.

Furthermore, existing telemedicine services struggle with scalability. As demand for remote consultations increases, healthcare providers face growing workloads, resulting in longer wait times and diminished quality of care. Traditional telemedicine solutions do not adequately leverage AI-driven automation to assist with patient intake, symptom evaluation, and triage, leaving physicians overburdened with routine consultations that could be efficiently managed through intelligent virtual assistants.

### SUMMARY OF THE INVENTION

This summary is provided to introduce a variety of concepts in a simplified form that is further disclosed in the detailed description of the embodiments. This summary is not intended for determining the scope of the claimed subject matter.

The embodiments provided herein disclose systems and methods for AI-driven telemedicine systems and methods to provide a consultation assistant that utilizes a combination of NLP, decision trees, machine learning algorithms, and integration with Electronic Health Records (EHR) to provide patients with preliminary consultations. The AI-Driven Telemedicine Consultation Assistant (AI-TCA) analyzes user-reported symptoms, medical history, and lab results to offer personalized diagnostic suggestions, health advice, and triage recommendations. The AI-TCA is designed to be integrated within telemedicine platforms, enabling seamless interaction and efficient patient triage before engaging with healthcare professionals.

To enhance the accuracy of its recommendations, the AI-TCA employs deep learning-based intent recognition within its NLP module. This enables the system to understand nuanced medical terminology and complex symptom descriptions, thereby improving diagnostic precision.

The decision tree module within AI-TCA is customized based on patient demographics, ensuring that diagnostic pathways consider factors such as age, gender, and pre-existing conditions. This personalized approach enhances the relevance of health recommendations for each patient.

To facilitate secure interactions, the AI-TCA supports voice and text-based input methods, allowing patients to communicate symptoms conveniently. This multimodal approach improves accessibility and user engagement.

To comply with regulatory requirements such as HIPAA, the EHR integration module securely encrypts patient data while ensuring seamless access to relevant medical history. This enhances the reliability of the system's diagnostic capabilities without compromising patient privacy.

In addition to providing preliminary diagnoses, the AI-TCA includes an alert mechanism for identifying urgent medical conditions. If symptoms indicate a potential emergency, the system notifies the patient and recommends immediate medical attention, ensuring timely intervention.

The system also supports multilingual interactions, allowing it to cater to diverse patient populations. This feature improves accessibility for non-English speakers and enhances healthcare inclusivity.

Through real-time symptom analysis and dynamic recommendation updates, the AI-TCA continuously refines its diagnostic outputs based on evolving patient data. This iterative learning approach ensures that the system remains up-to-date with the latest medical knowledge.

To further improve diagnostic accuracy, the AI-TCA maintains a database of anonymized diagnostic cases, which is used for training and validating its machine learning models. This data-driven approach enhances the robustness and reliability of its recommendations over time.

Additionally, the AI-TCA provides patients with links to relevant healthcare literature based on their conditions, empowering them with educational resources that promote informed decision-making about their health.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a system architecture diagram of the network infrastructure, according to some embodiments;

FIG. 2 illustrates a block diagram of the application program in communication with the computing system, according to some embodiments;

FIG. 3 illustrates a flowchart for a method of providing an AI-Driven Telemedicine Consultation Assistant, according to some embodiments; and

FIG. 4 illustrates a flowchart of the data flow used by the AI-Driven Telemedicine Consultation Assistant, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided herein relate to an AI-driven telemedicine systems and methods to provide a consultation assistant that utilizes a combination of NLP, decision trees, machine learning algorithms, and integration with Electronic Health Records (EHR) to provide patients with preliminary consultations. The AI-Driven Telemedicine Consultation Assistant (AI-TCA) analyzes user-reported symptoms, medical history, and lab results to offer personalized diagnostic suggestions, health advice, and triage recommendations. The AI-TCA is designed to be integrated within telemedicine platforms, enabling seamless interaction and efficient patient triage before engaging with healthcare professionals.

In some embodiments, the system includes an NLP module for processing user-inputted medical symptoms. This component employs deep learning models such as BERT or GPT-based architectures to recognize patient intent, extract relevant medical entities, and standardize text input. Unlike prior systems that rely solely on predefined keyword matching, the NLP module dynamically understands nuanced medical terminology and improves contextual awareness, addressing a long-standing limitation in existing telemedicine triage systems.

In some embodiments, the system includes a decision tree module which operates by analyzing structured data from the NLP module and mapping symptoms to potential diagnoses. This module incorporates a dynamic set of rules and branching pathways that consider various factors such as severity, duration, and co-occurring symptoms. The decision tree is continuously refined based on medical literature and patient outcome data to improve the accuracy of preliminary diagnoses. Unlike conventional rule-based decision trees, this module evolves dynamically based on real-world data. The decision tree adapts in real-time by incorporating updated medical literature, real-world patient data, and new findings, ensuring that diagnostic suggestions remain current and accurate.

In some embodiments, the system integrates Electronic Health Records (EHR) to retrieve patient medical history, past diagnoses, medications, and lab results. This integration enhances diagnostic accuracy by allowing the AI system to personalize recommendations based on historical health data. Unlike previous systems that provide only basic retrieval functions, the AI-TCA actively analyzes retrieved EHR data in real time to refine diagnostic outputs. Additionally, the system ensures compliance with HIPAA regulations through end-to-end encryption and secure authentication protocols, which is not adequately addressed in previous solutions. In sum, the integrations with EHRs allows the system to dynamically retrieve and process patient medical histories, past diagnoses, and lab results in real-time. This integration ensures personalized, up-to-date recommendations that reflect the patient's ongoing health journey.

In some embodiments, the system includes a machine learning module to refine diagnostic accuracy by continuously learning from prior patient interactions and medical datasets. The system utilizes supervised learning techniques to analyze vast amounts of structured and unstructured medical data. By training on real-world patient cases, the machine learning model improves pattern recognition and triage decisions. The machine learning module continuously learns from large, diverse medical datasets, enhancing its predictive accuracy and triage capabilities as more patient interactions and medical cases are processed.

In some embodiments, the system provides a user-friendly interface that enables patients to input symptoms and receive diagnostic recommendations. The UI supports both text-based and voice-based interactions to accommodate users with different accessibility needs. The conversational design of the interface allows seamless interaction while guiding patients through structured health assessments. Unlike generic chatbot-based solutions, the AI-TCA employs adaptive UI enhancements based on real-time patient responses to optimize user engagement. In sum, the adaptive UI responds to real-time patient inputs, adjusting its questions and prompts based on the patient's responses and medical context, offering a highly personalized and engaging experience.

During use, the patient inputs symptoms, queries, or medical history into the user interface. The NLP module processes the input, converting it into structured data and the decision tree module analyzes structured data and generates preliminary diagnoses. The HER system retrieves patient data to improve recommendation accuracy. The machine learning module then refines diagnosis outcomes based on prior interactions and medical datasets and the system presents a diagnosis, recommended care plan (or other actions, and triage advice to the patient. If an emergency is detected, the system notifies the patient and advises immediate medical attention. The system not only analyzes symptoms and HER data but also incorporates prioritization algorithms to flag high-risk conditions immediately. Emergency cases are flagged for rapid referral to a healthcare provided, with safeguards to ensure critical conditions are not missed.

The automated triage supports reduces patient wait times by automating initial triage and prioritizing high-risk cases. The system also provides enhanced decision-making and personalized healthcare in a scalable manner to reduce physician workload and increase accuracy in diagnosis and treatments plans.

The integration of machine learning-driven real-time EHR analysis significantly improves diagnostic efficiency by providing personalized, AI-enhanced triage assessments. The use of decision trees ensures that the system evolves over time based on real-world patient interactions, a feature not found in conventional systems. The real-time analysis of EHRs through machine learning enhances diagnostic decision-making by ensuring that each patient's medical history is continuously considered, leading to more accurate and timely triage diagnoses.

In some embodiments, the system may have the ability to process multimodal patient inputs including text, video, voice, etc. derived symptom reporting to enhance accessibility and usability, and improve the efficiency of telemedicine consultations.

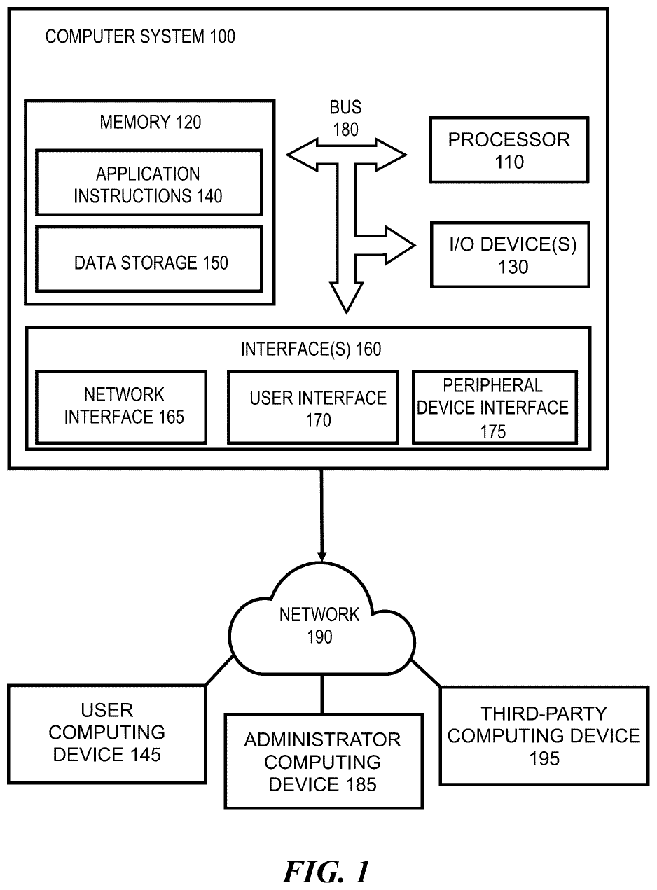

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computing device 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive). The computer system architecture ensures efficient operation by utilizing cloud-based resources for AI computations, allowing for seamless scaling and real-time processing of medical triage data.

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 150, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., Android, C, C++, C#, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a user computing device 145 an administrator computing device 185 and a third-party computing device 195 each in communication via the network 190. The administrator computing device 185 is utilized by an administrative user to moderate content and to perform other administrative functions. The third-party computing device 195 may be utilized by third parties to receive communications from the user computing device, transmit communications to the user via the network, and otherwise interact with the various functionalities of the system.

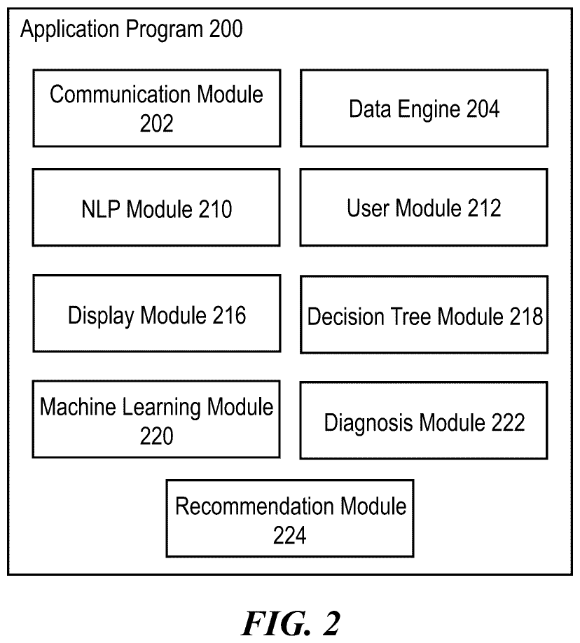

FIG. 2 illustrates an example computer architecture for the application program 200 operated via the computing system 100. The computer system 100 comprises several modules and engines configured to execute the functionalities of the application program 200, and a database engine 204 configured to facilitate how data is stored and managed in one or more databases. In particular, FIG. 2 is a block diagram showing the modules and engines needed to perform specific tasks within the application program 200.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of a communication module 202, a database engine 204, a natural language processing module 210, a user module 212, a display module 216, a decision tree module 218, a machine learning module 220, a diagnosis module 222, and a recommendation module 224.

In some embodiments, the communication module 202 is configured for receiving, processing, and transmitting a user command and/or one or more data streams. In such embodiments, the communication module 202 performs communication functions between various devices, including the user computing device 145, the administrator computing device 185, and a third-party computing device 195. In some embodiments, the communication module 202 is configured to allow one or more users of the system, including a third-party, to communicate with one another. In some embodiments, the communications module 202 is configured to maintain one or more communication sessions with one or more servers, the administrative computing device 185, and/or one or more third-party computing device(s) 195. In some embodiments, the communication module 202 allows each user to transmit and receive information which may be used by the system.

In some embodiments, a database engine 204 is configured to facilitate the storage, management, and retrieval of data to and from one or more storage mediums, such as the one or more internal databases described herein. In some embodiments, the database engine 204 is coupled to an external storage system. In some embodiments, the database engine 204 is configured to apply changes to one or more databases. In some embodiments, the database engine 204 comprises a search engine component for searching through thousands of data sources stored in different locations. The database engine 204 allows each user and module associated with the system to transmit and receive information stored in various databases.

In some embodiments, the database engine 204 may be in operable communication with an EHR database or system which provides electronic health record data including medical history related to the patient(s).

In some embodiments, the natural language processing module 210 processes user-inputted medical symptoms. This component employs deep learning models such as BERT or GPT-based architectures to recognize patient intent, extract relevant medical entities, and standardize text input. Unlike prior systems that rely solely on predefined keyword matching, the NLP module dynamically understands nuanced medical terminology and improves contextual awareness, addressing a long-standing limitation in existing telemedicine triage systems.

In some embodiments, the user module 212 facilitates the creation of a user account for the application system. The user module 212 may allow the user to input account information, establish user permissions, medical data, triage information, etc.

In some embodiments, the display module 216 is configured to display one or more graphic user interfaces, including, e.g., one or more user interfaces, one or more consumer interfaces, one or more video presenter interfaces, etc. In some embodiments, the display module 216 is configured to temporarily generate and display various pieces of information in response to one or more commands or operations. The various pieces of information or data generated and displayed may be transiently generated and displayed, and the displayed content in the display module 216 may be refreshed and replaced with different content upon the receipt of different commands or operations in some embodiments. In such embodiments, the various pieces of information generated and displayed in a display module 216 may not be persistently stored. The display module 216 provides alerts to the user device which can be viewed and acknowledged by the user.

In some embodiments, the decision tree module 218 analyzes structured data from the NLP module and mapping symptoms to potential diagnoses. This module incorporates a dynamic set of rules and branching pathways that consider various factors such as severity, duration, and co-occurring symptoms. The decision tree is continuously refined based on medical literature and patient outcome data to improve the accuracy of preliminary diagnoses. Unlike conventional rule-based decision trees, this module evolves dynamically based on real-world data.

In some embodiments, the machine learning module 220 refines diagnostic accuracy by continuously learning from prior patient interactions and medical datasets. The system utilizes supervised learning techniques to analyze vast amounts of structured and unstructured medical data. By training on real-world patient cases, the machine learning model improves pattern recognition and triage decisions. Unlike static AI models found in prior art, this module continuously adapts, reducing the likelihood of incorrect triage outcomes and significantly improving diagnostic efficiency.

In some embodiments, the diagnosis module 222 is capable of receiving information from the machine learning module 220 to generate a diagnosis related to the symptoms input by the user. This information is then used by the recommendation module 224 to generate a list of recommendations, a care plan, or other information which can be used to effect the user and their symptoms.

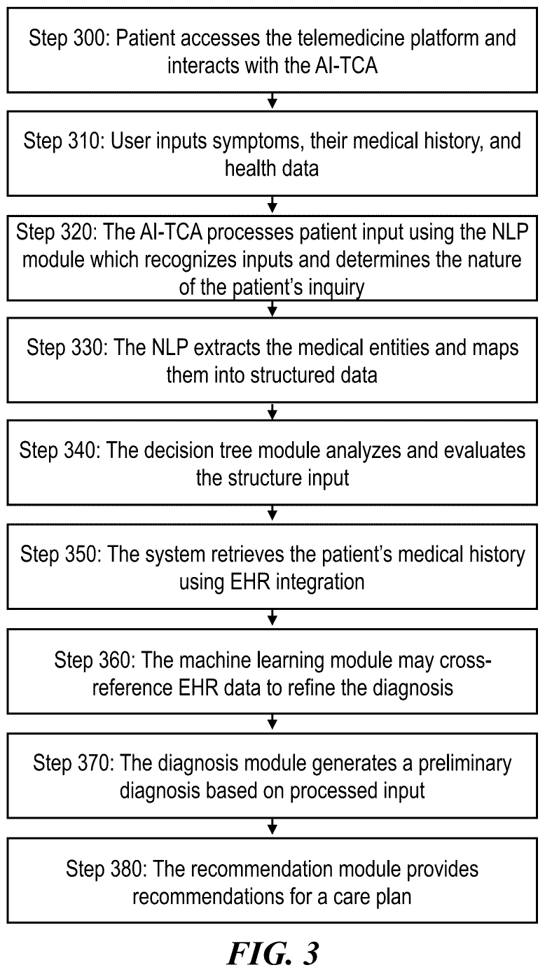

FIG. 3 illustrates a flowchart of a method for providing the AI-TCA using a telemedicine platform. In step 300, the patient accesses the telemedicine platform and interacts with the AI-TCA. In step 310, the user inputs symptoms, their medical history, and other relevant health data through the user interface (text or voice-based). In step 320, the AI-TCA processes patient input using the NLP module which may utilize techniques such as tokenization, lemmatization, and stopword removal. The NLP module then recognizes the inputs and determines the nature of the patient's inquiry. In step 330, the NLP module extracts the medical entities (i.e., symptoms, conditions, medications, etc.) and maps them into structured data. In step 340, the decision tree module analyzes and evaluates the structured input to determine potential conditions and may also perform a severity assessment to prioritize triage recommendations. In step 350, the system retrieves the patient's medical history using the EHR integration facilitated by the database engine. In step 360, the machine learning module may cross-reference EHR data to refine diagnosis accuracy and apply machine learning algorithms to adjust diagnostic accuracy based on patient history and population-wide datasets. In step 370, the diagnosis module generates a preliminary diagnosis based on processed input. In step 380, the recommendation module may provide recommendations for self-care instructions, general care plans, medications, lifestyle modifications, and other guidance.

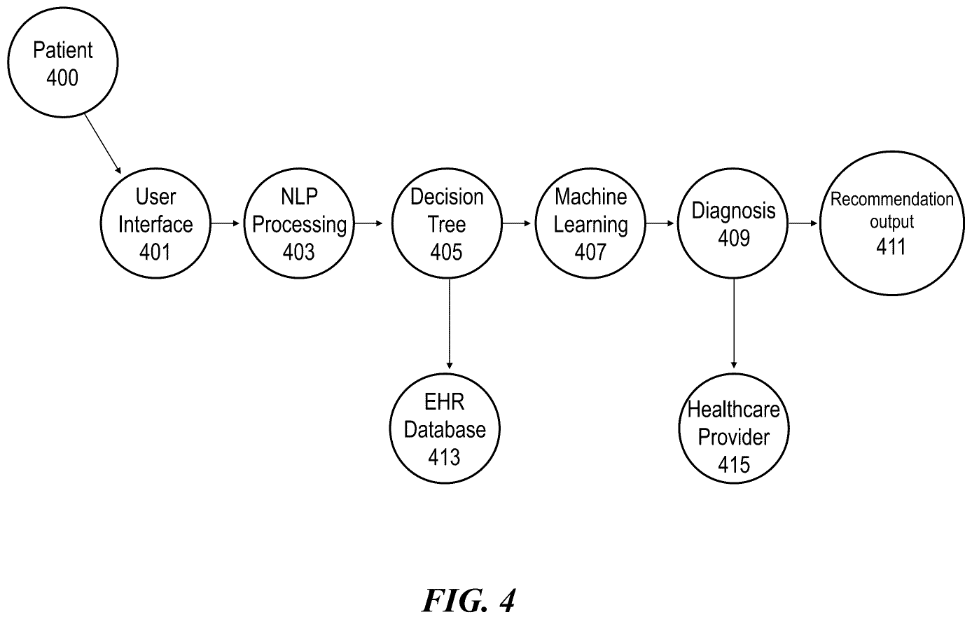

FIG. 4 illustrates a block diagram of the data flow within the system. The patient 400 utilizes the user interface 401 provided on a computing device to input information such as symptoms, medical history, and other information related to their health, current condition, etc. This information is passed through NLP processing 403, the decision tree 405, machine learning 407, and diagnosis steps 409 to generate the recommendation output 411. The diagnosis and/or recommendations are transmitted to an EHR database 413 and/or healthcare provider 415.

In some embodiment, the decision tree module is designed to dynamically evolve. It adapts by leveraging Machine Learning (ML) models, which are trained on vast datasets. As the AI-TCA processes more patient interactions, the machine learning models improve diagnostic accuracy and refine the decision-making process by identifying patterns from patient data, including symptoms, medical history, and lab results. This continuous learning allows the system to adapt based on real-world patient interactions and improve over time.

In some embodiments, the system integrates with the EHR database through an EHR integration module, which retrieves real-time patient data (such as medical history, past diagnoses, medications, and lab results). This integration is unique because it allows for personalized recommendations based on a patient's comprehensive health history. The decision tree and machine learning modules can access this data to refine diagnostic suggestions and provide more accurate triage recommendations.

The NLP module uses advanced deep learning models (e.g., BERT or GPT-based architectures) for intent recognition and entity extraction. When it encounters ambiguous or incomplete inputs, it can either ask clarifying questions or use fallback procedures, such as: Requesting more information from the patient to clarify the symptoms or queries; using probabilistic models to make reasonable assumptions and proceed with available information, though this may affect diagnostic confidence; and Providing generic advice when the input is unclear or insufficient to generate a specific recommendation.

While the core decision tree and machine learning algorithms are similar to existing models, the AI-TCA may have specific adaptations tailored for telemedicine applications. These adaptations could involve the way the system interacts with structured data from EHRs, how it processes real-time patient input, or how it combines decision trees with ML models to offer personalized health suggestions. Furthermore, integration with telemedicine platforms and the specific workflow of the system might bring unique enhancements to existing algorithms.

In some embodiments, the system includes alternative processes when it cannot access the HER or receive competent patient input. In the absence of EHR access, the AI-TCA may rely on default patient profiles (e.g., age, gender, and basic medical conditions) to offer general recommendations. The system may also use machine learning models to make decisions based on available patient inputs or symptoms, without the full medical history. If competent patient input is not received, the system may prompt for more details or provide generic advice. The system may also inform the user if it cannot confidently proceed without more information.

In some embodiments, the system may revert to automated triage protocols based on widely recognized guidelines (e.g., theECRI Guidelines or CDC Symptom Checkers).

In some embodiments, the system may provide a human-assisted option, where if the system is unable to provide a definitive answer, it can escalate the case to a human healthcare professional for further evaluation.

The system may utilize pre-recorded questions or prompts to gather more detailed information from patients when the system encounters incomplete or ambiguous inputs.

The system handles missing, incorrect, or conflicting data by following these steps:
Missing data: The system will attempt to fill in gaps using reasonable assumptions, or ask the patient for additional details. If this is not possible, the system will offer general guidance or escalate to a healthcare professional.
Incorrect data: If the system detects discrepancies in the input (e.g., symptoms that don't align with historical data), it may request clarification from the patient or recommend further tests.
Conflicting data: In cases of conflicting information (e.g., a patient's medical history suggesting one diagnosis, while symptoms suggest another), the system might:
Provide a probabilistic analysis, offering multiple possible diagnoses based on the conflicting inputs.
Request further confirmation from the patient regarding their current symptoms.
Flag the case for human review, ensuring that a healthcare professional can step in to resolve conflicts.

These methods ensure that the system remains effective and adaptable in handling real-world healthcare scenarios, especially when dealing with incomplete, ambiguous, or inconsistent data.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrase “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

What is claimed is:

1. An AI-driven telemedicine consultation system, comprising:
at least one user computing device in operable connection with a user network;
an application server in operable communication with the user network, the application server configured to host an application program for identifying and verifying individuals, the application program having a user interface module for providing access to the application program via the at least one user computing device;
a natural language processing module configured to process a plurality of medical symptoms;
a decision tree module to map the plurality of medical symptoms to one or more potential diagnoses;
an integration module to retrieve a medical history and one or more lab results from an electronic health record;
a machine learning module to refine the one or more potential diagnoses based on a plurality of stored patient interactions and a plurality of medical datasets; and
a user interface to facilitate a patient interaction and to output a recommendation.

2. The system of claim 1, wherein the NLP module employs deep learning models for enhanced intent recognition.

3. The system of claim 1, wherein the decision tree module is customized based on patient demographics.

4. The system of claim 1, further comprising multilingual support for diverse patient populations.

5. The system of claim 1, further comprising a database storing anonymized diagnostic cases for training future models.

6. The system of claim 1, wherein the output includes links to relevant healthcare articles based on patient conditions.

7. A system for AI-assisted patient triage in telemedicine, the system comprising:
a user interface for receiving a plurality of patient symptoms and a plurality of medical history data;
a natural language processing module trained for intent recognition and entity extraction;
a medical decision support engine utilizing a plurality of decision trees for generation of a diagnoses;
an application programming interface for integration with an electronic health record;
a machine learning muddle for refining a plurality of triage outcomes and a plurality of predictive analytics.

8. The system of claim 7, wherein the user input interface supports voice and text-based interactions.

9. The system of claim 7, wherein the EHR integration module securely encrypts patient data to comply with HIPAA regulations.

10. The system of claim 7, wherein the machine learning model is retrained based on patient feedback.

11. The system of claim 7, further comprising an alert mechanism for urgent medical conditions based on patient input.

12. A method for providing automated telemedicine consultations, the method
comprising the steps of:
receiving, an input comprising a plurality of patient symptoms and a medical history through a user interface;
processing the input using a natural language processing module to identify one or more symptoms;
generating structured data based on the one or more identified symptoms;
using a decision tree to assess one or more potential conditions based on the one or more identified symptoms;
retrieving the medical history from an electronic health record system;
refining the one or more potential conditions using a machine learning algorithm;
presenting one or more recommendations, triage advice, and a care plan to a patient.

13. The method of claim 12, wherein patient input is preprocessed using tokenization and stopword removal.

14. The method of claim 12, wherein health recommendations include self-care suggestions and emergency triage indicators.

15. The method of claim 12, further comprising real-time symptom analysis and recommendation updates.

16. The method of claim 12, further comprising a chatbot interface for guiding patients through symptom entry.

17. The method of claim 12, wherein lab result interpretation is prioritized for real-time updates in triage recommendations.
